(19) United States
(12) Patent Application Publication
(10) Pub. No.: US 2024/0277788 A1
(43) Pub. Date: Aug. 22, 2024

(54) COMPOSITIONS FOR INCREASING RESILIENCE TOWARDS BACTERIAL INFECTIONS

(71) Applicant: Chr. Hansen A/S, Hoersholm (DK)

(72) Inventors: Lea Hübertz Birch Hansen, Hoersholm (DK); Beatrice Nielsen, Hoersholm (DK)

(73) Assignee: Chr. Hansen A/S, Hoersholm (DK)

(21) Appl. No.: 18/570,816

(22) PCT Filed: Jun. 16, 2022
(86) PCT No.: PCT/EP2022/066487

§ 371 Date: Dec. 15, 2023

(30) Foreign Application Priority Data
Jun. 18, 2021 (EP) 21180190.7

(51) Int. Cl. A61K 35/747; A61K 35/00; A61K 35/744; A61K 35/745; A61P 31/04

(52) U.S. Cl. CPC A61K 35/747; A61K 35/744; A61K 35/745; A61P 31/04; A61K 2035/115

(57) ABSTRACT

The present invention provides evidence that administration of a probiotic product to newborn piglets and during suckling makes the piglets more resilient towards an ETEC challenge post-weaning and after cessation of the probiotic administration. The pigs administered with probiotics pre-weaning were faster at overcoming the ETEC infection by which ETEC counts in feces were significantly faster reduced compared with non-probiotic supplemented pigs. Keywords: Pig, ETEC F18, Probiotic, post-weaning diarrhea, microbiome, immunity

### FIELD OF THE INVENTION

The present invention relates to compositions containing probiotic bacteria for use in methods for increasing the resilience to an infection by a pathogenic bacterium in a mammal. The present invention is particularly related to the field of animal husbandry.

### BACKGROUND OF THE INVENTION

Many studies have investigated the direct effect of administering probiotic bacteria post-weaning in feed to nursery pigs and pigs challenged with Enterotoxigenic Escherichia coli (ETEC).

Few studies have been carried out investigating the effect of probiotic administration to newborn or suckling piglets. Pre-weaning, the suckling piglet is still protected by maternal antibodies. Post-weaning, the piglet undergoes many changes such as transport, mixing, change of diet and environment, all which make the piglet more vulnerable and susceptible to infectious disease such as ETEC infection.

The present invention aims to provide compositions that help protect young mammals from severe infections by pathogenic bacteria, such as ETEC F18, post-weaning.

### SUMMARY OF THE INVENTION

To the knowledge of the inventors, no studies have assessed the effect of administration of a composition of probiotic bacteria pre-weaning on piglets' ability to overcome pathogenic challenge post-weaning and after cessation of the probiotic administration.

The novel finding of the present invention is the beneficial effect of probiotic bacteria after cessation of the administration, i.e., that the resilience of piglets to severe infections can be increased post-weaning.

The presently disclosed compositions and their administration during the pre-weaning period may reduce the need to administer zinc oxide and antibiotics to treat severe infections in post-weaned mammals. Zinc oxide is considered a pollutant and excessive use of antibiotics is associated with the acceleration of the incidence of antibiotic resistance in pathogenic bacterial strains. Thus, there is a need to reduce severe infections in post-weaned mammals without excessively relying on zinc oxide or antibiotics.

The present invention addresses this need by providing a composition comprising probiotic bacteria. The composition may be for use in a method of increasing resilience against infection by a pathogenic bacterium in a mammalian subject, wherein the method comprises administering the composition to the subject in the pre-weaning period.

Other aspects of the invention are provided in the claims and will be discussed in detail below.

### FIGURES

FIG. 1 discloses the percentage of pigs with diarrhea (score >3) following oral administration on day 1-2 post-weaning of NaCl (Negative Control group •); of ETEC F18 (Positive Control group ▴); and of ETEC F18 and probiotic administration pre-weaning (Probiotic group ▪).

FIG. 2 discloses the percentage of pigs with detectable numbers of ETEC F18 in feces following oral administration on day 1-2 post-weaning of NaCl (Negative Control group •); of ETEC F18 (Positive Control group ▴); and of ETEC F18 and probiotic administration pre-weaning (Probiotic group ▪). ETEC F18 in feces was detected by enumerating on blood agar plates and serotyping. The detection limit was 5 log CFU/g.

FIG. 3 discloses the percentage of pigs detectable with the est-II gene in feces following oral administration on day 1-2 post-weaning of NaCl (Negative Control group •); of ETEC F18 (Positive Control group ▴); and of ETEC F18 and probiotic administration pre-weaning (Probiotic group ▪). The est-II gene encoding for the STb toxin was quantified by qPCR. Detection limit was 5 log cells/g.

FIG. 4 discloses the principal coordinates analysis (PCoA) of Bray-Curtis dissimilarity between the Control (•) and Probiotic group (▴) on day 35. Bray-Curtis distance metrics were used to compare the composition of the microbiota between the two treatment groups in a) small intestinal mucosa, b) digesta of the proximal colon, and feces in round c) 1, d) 2, and e) 3. Nested permutational multivariate analysis of variance (PERMANOVA) on Bray-Curtis distance metrics with sow ID nested with treatment group was carried out using 999 permutations to test for significance of clustering pattern. P-values for the effect of probiotic treatment are illustrated. P<0.05 was considered significant whereas P<0.10 was considered as a statistical tendency.

FIG. 5 discloses the fold change of small intestinal mucosal gene expression in Control and Probiotic pigs on day 23-24 and day 35-36. * indicates statistical significance between treatment groups (P<0.05), ▪ indicates a trend towards statistical significance (P<0.10), determined by mixed model. Number of samples: Control d23−24=22, d35−36=22. Probiotic d23−24=20, d35−36=18.

FIG. 6 discloses the effect of early probiotic inoculation on percent pens with diarrhea (score 3 and 4). Control ▴ (n=12 pens), Probiotic • (n=12 pens). The dotted line illustrates the day of weaning. * indicates statistical significance (P<0.05), ▪ indicates a trend towards statistical significance (P<0.10).

FIG. 7 discloses the relative transepithelial electrical resistance (TEER) across differentiated Caco-2 cell monolayers exposed to ETEC F4 in the presence or absence of E. faecium. The results are presented as mean f standard deviation (SD) (n=3).

FIG. 8 discloses the released IL-12 from dendritic cells after stimulation with E. faecium and two Lactobacillus strains.

FIG. 9 discloses the released IL-10 from dendritic cells after stimulation with E. faecium and two Lactobacillus strains.

### DETAILED DESCRIPTION OF THE INVENTION

### Definitions

The term “Direct-Fed Microbial” or “DFM” refers to compositions comprising live bacteria including spores which, when administered in adequate amounts, confer a benefit, such as improved digestion or health, on the host. The bacteria may be freeze-dried or lyophilized.

Within the context of the present invention, the expression “mammalian subject” refers to a human infant or other young mammal. In some embodiments, the mammalian subject is an infant (human, monkey, chimpanzee or gorilla), kitten, puppy, piglet, kit (rabbit or ferret), pup (gerbil, hamster, guinea pig, rat, seal, meerkat, lemur, bat or mouse), foal (horse or donkey), calf (cow, yak, elephant, dugong, manatee, rhinoceros, giraffe or camel), lamb, kid (goat), cria (alpaca or llama), cub (lion, tiger or bear) or joey (kangaroo or koala). Preferably, the mammalian subject is a piglet.

The expression “increased resilience against infection by a pathogenic bacterium” refers to a decrease in disease severity when a mammalian subject is challenged with a pathogenic bacterium. A decrease in disease severity may be characterized by:
(i) a decrease in the number of days that a group of mammalian subjects which have been treated with the compositions of the present invention suffer from diarrhea when compared to a comparable group of mammalian subjects which have not been treated; and/or
(ii) a reduction in fecal shedding of the bacterium and/or a toxin produced by the bacterium in a group of mammalian subjects which have been treated with the composition of the present invention when compared to a comparable group of untreated mammalian subjects.

In the case of an ETEC F18 infection, a decrease in the number of days with diarrhea as well as lower fecal shedding of ETEC F18 and Stb toxin was observed in the treated group when compared to the untreated group.

In the present invention, the expression “pathogenic bacterium” refers to any bacterial strain that can cause a disease by infecting the gastrointestinal tract of a mammalian subject. In some embodiments, the pathogenic bacterium is a pathogenic E. coli strain, such as an ETEC strain. Preferably, the pathogenic bacterium is ETEC F4 or ETEC F18 (see, for example, Luise et al., 2019. J Anim Sci Biotechnol. 10:53). More preferably, the pathogenic bacterium is ETEC F18.

The term “probiotic” refers to any composition which, when applied to animal or human, beneficially affects the host (FAO/WHO (2001) Health and Nutritional Properties of Probiotics in Food including Powder Milk with Live Lactic Acid Bacteria. Report of a Joint FAO/WHO Expert Consultation on Evaluation of Health and Nutritional Properties of Probiotics in Food Including Powder Milk with Live Lactic Acid Bacteria). The expression “probiotic bacteria” encompasses cultures of live bacteria, dead bacteria, fragments of bacteria and extracts or supernatants of bacterial cultures. Probiotic bacterial compositions of the present invention can be preferably administered as a Direct-Fed Microbial (DFM).

The term “weaning” refers to the process of introducing an infant human or another young mammal (e.g., a piglet) to what will be its adult diet while withdrawing the supply of its mother's milk or alternatives thereof (e.g., infant formula). This process may be gradual or abrupt. Thus, the pre-weaning period refers to the period directly after birth and before the weaning starts and the post-weaning period refers to the period directly after the mother's milk (or a suitable alternative) has been withdrawn from the mammalian subject's diet.

### Compositions

Post-weaning diarrhea (PWD) primarily occurs during the first two weeks post-weaning where pigs are challenged with several stressors. Several risk factors influence the development of disease and include separation from the sow, change of diet, mixing with unfamiliar pigs and new housing conditions. ETEC is the most common cause of PWD, and ETEC with fimbria F18 and F4 are the most common pathogenic strains among ETEC causing PWD. In Denmark, selective breeding for ETEC F4 resistance has been carried out since 2003. However, breeding directed towards single genes for multifactorial diseases as PWD is not a successful strategy. Meanwhile, PWD caused by ETEC F18 infection has been an emerging challenge during the last decade.

A resilient microbiome and a well-functioning immune system are prerequisites for the pig to resist PWD. Several studies have shown that the microbiome of the pig is unstable after the weaning transition. Since one of the important roles of the gut microbiome is to protect the host against pathogens, responding to an external disturbance such as proliferation of ETEC can be a big challenge during the event of weaning. At the same time, an abrupt withdrawal of maternal milk at weaning removes the passive immunity and leaves the pig with a still immature immune system, making the pig more vulnerable to infections.

Interventions to prevent PWD are often implemented post-weaning. These can include the use of feed additives such as organic acids, pre- and probiotics, or enzymes. Especially administration of probiotics as a preventive mean towards PWD has been investigated. Probiotics are defined as “live microorganisms that, when administered in adequate amounts, confer a health benefit on the host”. Even though probiotics are proven to possess mechanisms of action beneficial to the host during stressful conditions such as ETEC infections in pigs, there are inconsistent results from studies looking at administration of probiotics in nursery feed as a direct alternative to medical zinc oxide.

One of the reasons for the inconsistent results could be that intervention immediately before the event of dysbiosis caused by weaning does not allow the probiotic time to exert its effects. According to Dou et al., 2017. PLoS One. 12(1):e0169851, it is possible to discriminate between pigs susceptible to PWD already seven days after birth based on their gut microbiota composition.

According to the inventors of the present invention, this indicates that early life colonization pattern seems to have a great impact on whether the pig is prone to suffer from PWD. Therefore, it is plausible that intervention with beneficial microbes early in life during a so-called “window of opportunity” would be a promising method to improve the intestinal microbial colonization pattern. This early intervention would then increase the chance of establishing a homeostatic ecosystem and improve the immunological development, possibly promoting maturity of these systems and increasing robustness of the host to withstand infectious disease post-weaning.

Thus, in a first aspect, the present invention provides a composition comprising probiotic bacteria.

The composition of probiotic bacteria of the present disclosure may comprise one, two, three, four, five, six, seven, eight, nine, ten or even more bacterial strains.

In one embodiment the composition of probiotic bacteria of the present disclosure comprises a bacterial strain of the genus Enterococcus, such as Enterococcus faecium. The characteristics of Enterococcus faecium are described in Schleifer & Klipper-Bälz, 1984. Int J Syst Evol. 34(1):31-34. A representative 16S rDNA sequence of E. faecium is provided as SEQ ID NO: 1:

TAATACATGCAAGTCGAACGCTTCTTTTTCCACCGGAGCTTGCTC
CACCGGAAAAAGAGGAGTGGCGAACGGGTGAGTAACACGTGGGTA
ACCTGCCCATCAGAAGGGGATAACACTTGGAAACAGGTGCTAATA
CCGTATAACAATCGAAACCGCATGGTTTTGATTTGAAAGGCGCTT
TCGGGTGTCGCTGATGGATGGACCCGCGGTGCATTAGCTAGTTGG
TGAGGTAACGGCTCACCAAGGCCACGATGCATAGCCGACCTGAGA
GGGTGATCGGCCACATTGGGACTGAGACACGGCCCAAACTCCTAC
GGGAGGCAGCAGTAGGGAATCTTCGGCAATGGACGAAAGTCTGAC
CGAGCAACGCCGCGTGAGTGAAGAAGGTTTTCGGATCGTAAAACT
CTGTTGTTAGAGAAGAACAAGGATGAGAGTAACTGTTCATCCCTT
GACGGTATCTAACCAGAAAGCCACGGCTAACTACGTGCCAGCAGC
CGCGGTAATACGTAGGTGGCAAGCGTTGTCCGGATTTATTGGGCG
TAAAGCGAGCGCAGGCGGTTTCTTAAGTCTGATGTGAAAGCCCCC
GGCTCAACCGGGGAGGGTCATTGGAAACTGGGAGACTTGAGTGCA
GAAGAGGAGAGTGGAATTCCATGTGTAGCGGTGAAATGCGTAGAT
ATATGGAGGAACACCAGTGGCGAAGGCGGCTCTCTGGTCTGTAAC
TGACGCTGAGGCTCGAAAGCGTGGGGAGCAAACAGGATTAGATAC
CCTGGTAGTCCACGCCGTAAACGATGAGTGCTAAGTGTTGGAGGG
TTTCCGCCCTTCAGTGCTGCAGCTAACGCATTAAGCACTCCGCCT
GGGGAGTACGACCGCAAGGTTGAAACTCAAAGGAATTGACGGGGG
CCCGCACAAGCGGTGGAGCATGTGGTTTAATTCGAAGCAACGCGA
AGAACCTTACCAGGTCTTGACATCCTTTGACCACTCTAGAGATAG
AGCTTCCCCTTCGGGGGCAAAGTGACAGGTGGTGCATGGTTGTCG
TCAGCTCGTGTCGTGAGATGTTGGGTTAAGTCCCGCAACGAGCGC
AACCCTTATTGTTAGTTGCCATCATTCAGTTGGGCACTCTAGCAA
GACTGCCGGTGACAAACCGGAGGAAGGTGGGGATGACGTCAAATC
ATCATGCCCCTTATGACCTGGGCTACACACGTGCTACAATGGGAA
GTACAACGAGTTGCGAAGTCGCGAGGCTAAGCTAATCTCTTAAAG
CTTCTCTCAGTTCGGATTGCAGGCTGCAACTCGCCTGCATGAAGC
CGGAATCGCTAGTAATCGCGGATCAGCACGCCGCGGTGAATACGT
TCCCGGGCCTTGTACACACCGCCCGTCACACCACGAGAGTTTGTA
ACACCCGAAGTCGGTGAGGTAACCTTTTGGAGCCAGCCGCCTAAG
GTGGGATAGATGATTGGGGTGAAGTCGTAACAAGGTA

A bacterium may be identified as belonging to the species Enterococcus faecium if it comprises a 16S rDNA sequence that has at least 97% (preferably at least 99%) sequence identity with SEQ ID NO: 1.

In one embodiment the composition of probiotic bacteria of the present disclosure comprises a bacterial strain of the genus Lactobacillus, such as Lactobacillus acidophilus, Lactobacillus animalis, Lactobacillus brevis, Lactobacillus buchneri, Lactobacillus casei, Lactobacillus delbrueckii, Lactobacillus diolivorans, Lactobacillus fermentum, Lactobacillus gasseri, Lactobacillus johnsonii, Lactobacillus plantarum, Lactobacillus paracasei, and Lactobacillus reuteri.

In one embodiment the composition of probiotic bacteria of the present disclosure comprises a bacterial strain of the genus Lacticaseibacillus such as Lacticaseibacillus rhamnosus. In some embodiments, the composition comprises a bacterium of the species Lacticaseibacillus rhamnosus.

The characteristics of Lacticaseibacillus rhamnosus, which was formerly known as Lactobacillus rhamnosus, are described in Zheng et al., 2020. Int J Syst Evol Microbiol. 70(4):2782-2858. A representative 16S rDNA sequence of L. rhamnosus is provided as SEQ ID NO: 2:

AGAGTTTGATCATGGCTCAGGATGAACGCTGGCGGCGTGCCTAAT
ACATGCAAGTCGAACGAGTTCTGATTATTGAAAGGTGCTTGCATC
TTGATTTAATTTTGAACGAGTGGCGGACGGGTGAGTAACACGTGG
GTAACCTGCCCTTAAGTGGGGGATAACATTTGGAAACAGATGCTA
ATACCGCATAAATCCAAGAACCGCATGGTTCTTGGCTGAAAGATG
GCGTAAGCTATCGCTTTTGGATGGACCCGCGGCGTATTAGCTAGT
TGGTGAGGTAACGGCTCACCAAGGCAATGATACGTAGCCGAACTG
AGAGGTTGATCGGCCACATTGGGACTGAGACACGGCCCAAACTCC
TACGGGAGGCAGCAGTAGGGAATCTTCCACAATGGACGCAAGTCT
GATGGAGCAACGCCGCGTGAGTGAAGAAGGCTTTCGGGTCGTAAA
ACTCTGTTGTTGGAGAAGAATGGTCGGCAGAGTAACTGTTGTCGG
CGTGACGGTATCCAACCAGAAAGCCACGGCTAACTACGTGCCAGC
AGCCGCGGTAATACGTAGGTGGCAAGCGTTATCCGGATTTATTGG
GCGTAAAGCGAGCGCAGGCGGTTTTTTAAGTCTGATGTGAAAGCC
CTCGGCTTAACCGAGGAAGTGCATCGGAAGCTGGAAAACTTGAGT
GCAGAAGAGGACAGTGGAACTCCATGTGTAGCGGTGAAATGCGTA
GATATATGGAAGAACACCAGTGGCGAACGCGGCTGTCTGGTCTGT
AACTGACGCTGAGGCTCGAAAGCATGGGTAGCGAACAGGATTAGA
TACCCTGGTAGTCCATGCCGTAAACGATGAATGCTAGGTGTTGGA
GGGTTTCCGCCCTTCAGTGCCGCAGCTAACGCATTAAGCATTCCG
CCTGGGGAGTACGACCGCAAGGTTGAAACTCAAAGGAATTGACGG
GGGCCCGCACAAGCGGTGGAGCATGTGGTTTAATTCGAAGCAACG
CGAAGAACCTTACCAGGTCTTGACATCTTTTGATCACCTGAGAGA
TCAGGTTTCCCCTCCGGGGGCAAAATGACAGGTGGTGCATGGTTG
TCGTCAGCTCGTGTCGTGAGATGTTGGGTTAAGTCCCGCAACGAG
CGCAACCCTTATGACTAGTTGCCAGCATTTAGTTGGGCACTCTAG
TAAGACTGCCGGTGACAAACCGGAGGAAGGTGGGGATGACGTCAA
ATCATCATGCCCCTTATGACCTGGGCTACACACGTGCTACAATGG
ATGGTACAACGAGTTGCGAGACCGCGAGGTCAAGCTAATCTCTTA
AAGCCATTCTCAGTTCGGACTGTAGGCTGCAACTCGCCTACACGA
AGTCGGAATCGCTAGTAATCGCGGATCAGCACGCCGCGGTGAATA
CGTTCCCGGGCCTTGTACACACCGCCCGTCACACCATGAGAGTTT
GTAACACCCGAAGCCGGTGGCGTAACCCTTTTAGGGAGCGAGCCG
TCTAAGGTGGGACAAATGATTAGGGTGAAGTCGTAACAAGGTAGC
CGTAGGAGAACCTGCGGCTGGATCACCTCCTTTCT

A bacterium may be identified as belonging to the species Lacticaseibacillus rhamnosus if it comprises a 16S rDNA sequence that has at least 97% (preferably at least 99%) sequence identity with SEQ ID NO: 2.

In one embodiment the composition of probiotic bacteria of the present disclosure comprises a bacterial strain of the genus Bifidobacterium, such as Bifidobacterium animalis, Bifidobacterium breve, Bifidobacterium infantis, or Bifidobacterium longum. In some embodiments, the composition comprises a bacterium of the species Bifidobacterium breve.

The characteristics of Bifidobacterium breve are described in Reuter, 1963. Zentralbl Bakteriol Parasitenkd Infektionskr Hyg Abt 1 Orig. 191:486-507. A representative 16S rDNA sequence of B. breve is provided as SEQ ID NO: 3:

TTCGATTCTGGCTCAGGATGAACGCTGGCGGCGTGCTTAACACAT
GCAAGTCGAACGGGATCCATCGGGCTTTGCTTGGTGGTGAGAGTG
GCGAACGGGTGAGTAATGCGTGACCGACCTGCCCCATGCACCGGA
ATAGCTCCTGGAAACGGGTGGTAATGCCGGATGCTCCATCACACC
GCATGGTGTGTTGGGAAAGCCTTTGCGGCATGGGATGGGGTCGCG
TCCTATCAGCTTGATGGCGGGGTAACGGCCCACCATGGCTTCGAC
GGGTAGCCGGCCTGAGAGGGCGACCGGCCACATTGGGACTGAGAT
ACGGCCCAGACTCCTACGGGAGGCAGCAGTGGGGAATATTGCACA
ATGGGCGCAAGCCTGATGCAGCGACGCCGCGTGAGGGATGGAGGC
CTTCGGGTTGTAAACCTCTTTTGTTAGGGAGCAAGGCACTTTGTG
TTGAGTGTACCTTTCGAATAAGCACCGGCTAACTACGTGCCAGCA
GCCGCGGTAATACGTAGGGTGCAAGCGTTATCCGGAATTATTGGG
CGTAAAGGGCTCGTAGGCGGTTCGTCGCGTCCGGTGTGAAAGTCC
ATCGCTTAACGGTGGATCCGCGCCGGGTACGGGCGGGCTTGAGTG
CGGTAGGGGAGACTGGAATTCCCGGTGTAACGGTGGAATGTGTAG
ATATCGGGAAGAACACCAATGGCGAAGGCAGGTCTCTGGGCCGTT
ACTGACGCTGAGGAGCGAAAGCGTGGGGAGCGAACAGGATTAGAT
ACCCTGGTAGTCCACGCCGTAAACGGTGGATGCTGGATGTGGGGC
CCGTTCCACGGGTTCCGTGTCGGAGCTAACGCGTTAAGCATCCCG
CCTGGGGAGTACGGCCGCAAGGCTAAAACTCAAAGAAATTGACGG
GGGCCCGCACAAGCGGCGGAGCATGCGGATTAATTCGATGCAACG
CGAAGAACCTTACCTGGGCTTGACATGTTCCCGACGATCCCAGAG
ATGGGGTTTCCCTTCGGGGCGGGTTCACAGGTGGTGCATGGTCGT
CGTCAGCTCGTGTCGTGAGATGTTGGGTTAAGTCCCGCAACGAGC
GCAACCCTCGCCCCGTGTTGCCAGCGGATTGTGCCGGGAACTCAC
GGGGGACCGCCGGGGTTAACTCGGAGGAAGGTGGGGATGACGTCA
GATCATCATGCCCCTTACGTCCAGGGCTTCACGCATGCTACAATG
GCCGGTACAACGGGATGCGACAGTGCGAGCTGGAGCGGATCCCTG
AAAACCGGTCTCAGTTCGGATCGCAGTCTGCAACTCGACTGCGTG
AAGGCGGAGTCGCTAGTAATCGCGAATCAGCAACGTCGCGGTGAA
TGCGTTCCCGGGCCTTGTACACACCGCCCGTCAAGTCATGAAAGT
GGGCAGCACCCGAAGCCGGTGGCCTAACCCCTTGCGGGAGGGAGC
CGTCTAAGGTGAGGCTCGTGATTGGGACTAAGTCGTAACAAGGTA

A bacterium may be identified as belonging to the species Bifidobacterium breve if it comprises a 16S rDNA sequence that has at least 97% (preferably at least 99%) sequence identity with SEQ ID NO: 3.

In some embodiments, the composition comprises a bacterium of the species Bifidobacteriumlongum. The characteristics of Bifidobacterium longum are described in Reuter, 1963. Zentralbl Bakteriol Parasitenkd Infektionskr Hyg Abt 1 Orig. 191:486-507.

In some embodiments, the composition comprises a bacterium of the subspecies Bifidobacterium longum subsp. infantis.

The characteristics of Bifidobacterium longum subsp. infantis are described in Mattarelli et al., 2008. Int J Syst Evol Microbiol. 58(Pt 4):767-72. A representative 16S rDNA sequence of Bifidobacterium longum subsp. infantis is provided as SEQ ID NO: 4:

GGCTCAGGATGAACGCTGGCGGCGTGCTTAACACATGCAAGTCGA
ACGGGATCCATCGGGCTTTGCTTGGTGGTGAGAGTGGCGAACGGG
TGAGTAATGCGTGACCGACCTGCCCCATACACCGGAATAGCTCCT
GGAAACGGGTGGTAATGCCGGATGTTCCAGTTGATCGCATGGTCT
TCTGGGAAAGCTTTCGCGGTATGGGATGGGGTCGCGTCCTATCAG
CTTGACGGCGGGGTAACGGCCCACCGTGGCTTCGACGGGTAGCCG
GCCTGAGAGGGCGACCGGCCACATTGGGACTGAGATACGGCCCAG
ACTCCTACGGGAGGCAGCAGTGGGGAATATTGCACAATGGGCGCA
AGCCTGATGCAGCGACGCCGCGTGAGGGATGGAGGCCTTCGGGTT
GTAAACCTCTTTTATCGGGGAGCAAGCGTGAGTGAGTTTACCCGT
TGAATAAGCACCGGCTAACTACGTGCCAGCAGCCGCGGTAATACG
TAGGGTGCAAGCGTTATCCGGAATTATTGGGCGTAAAGGGCTCGT
AGGCGGTTCGTCGCGTCCGGTGTGAAAGTCCATCGCTTAACGGTG
GATCCGCGCCGGGTACGGGCGGGCTTGAGTGCGGTAGGGGAGACT
GGAATTCCCGGTGTAACGGTGGAATGTGTAGATATCGGGAAGAAC
ACCAATGGCGAAGGCAGGTCTCTGGGCCGTTACTGACGCTGAGGA
GCGAAAGCGTGGGGAGCGAACAGGATTAGATACCCTGGTAGTCCA
CGCCGTAAACGGTGGATGCTGGATGTGGGGCCCGTTCCACGGGTT
CCGTGTCGGAGCTAACGCGTTAAGCATCCCGCCTGGGGAGTACGG
CCGCAAGGCTAAAACTCAAAGAAATTGACGGGGGCCCGCACAAGC
GGCGGAGCATGCGGATTAATTCGATGCAACGCGAAGAACCTTACC
TGGGCTTGACATGTTCCCGACGATCCCAGAGATGGGGTTTCCCTT
CGGGGGGGGTTCACAGGTGGTGCATGGTCGTCGTCAGCTCGTGTC
GTGAGATGTTGGGTTAAGTCCCGCAACGAGCGCAACCCTCGCCCC
GTGTTGCCAGCGGATTGTGCCGGGAACTCACGGGGGACCGCCGGG
GTTAACTCGGAGGAAGGTGGGGATGACGTCAGATCATCATGCCCC
TTACGTCCAGGGCTTCACGCATGCTACAATGGCCGGTACAACGGG
ATGCGACGCGGCGACGCGGAGCGGATCCCTGAAAACCGGTCTCAG
TTCGGATCGCAGTCTGCAACTCGACTGCGTGAAGGCGGAGTCGCT
AGTAATCGCGAATCAGCAACGTCGCGGTGAATGCGTTCCCGGGCC
TTGTACACACCGCCCGTCAAGTCATGAAAGTGGGCAGCACCCGAA
GCCGGTGGCCTAACCCCTTGTGGGATGGAGCCGTCTAAGGTGAGG
CTCGTGATTGGGACTAAGTCGTAACAAGGTAGCCGTACCGGAAGG
TGCGGCT

A bacterium may be identified as belonging to the subspecies Bifidobacterium longum subsp. infantis if it comprises a 16S rDNA sequence that has at least 97% (preferably at least 99%) sequence identity with SEQ ID NO: 4.

References to a percentage sequence identity between two nucleotide sequences means that, when aligned, that percentage of nucleotides are the same in comparing the two sequences. This alignment and the percent homology or sequence identity can be determined using the approach described in Drancourt et al., 2000. J Clin Microbiol. 38(10):3623-30, i.e., using the BLOSUM 62 matrix with default parameters including a gap existence cost of 11, a cost-per-residue gap of 1 and a lambda ratio of 0.85.

In one embodiment the composition of probiotic bacteria of the present disclosure comprises a bacterial strain of the genus Bacillus, such as of the species Bacillus altitudinis, Bacillus amyloliquefaciens, e.g. Bacillus amyloliquefaciens subsp. amyloliquefaciens or Bacillus amyloliquefaciens subsp. plantarum, Bacillus atrophaeus, Bacillus licheniformis, Bacillus megaterium, Bacillus methylotrophicus, Bacillus mojavensis, Bacillus pumilus, Bacillus safensis, Bacillus simplex, Bacillus stratosphericus, Bacillus subtilis, Bacillus siamensis, Bacillus vallismortis, Bacillus velezensis, or Bacillus tequilensis.

In some embodiments, the composition comprises a bacterium:
(a) belonging to the species Enterococcus faecium;
(b) belonging to the subspecies Bifidobacterium longum subsp. infantis;
(c) belonging to the species Bifidobacterium breve; and/or
(d) belonging to the species Lacticaseibacillus rhamnosus.

In some embodiments, the compositions comprises no more than 1 to 20 bacterial species. In other words, while the composition may comprise other components such as cryoprotectants or lyoprotectants, the composition does not contain any other bacterial species or comprises only de minimis or biologically irrelevant amounts of other bacterial species. In some embodiments, the composition comprises no more than 1, 2, 3, 4, 5, 6, 7, 8, 9, 10, 11, 12, 13, 14, 15, 16, 17, 18, 19 or 20 bacterial species. In some embodiments, the composition comprises no more than 2 to 10 or 2 to 5 bacterial species. Preferably, the composition comprises no more than 4 or 5 bacterial species.

In some embodiments, the composition comprises at least 2, 3 or 4 bacterial species.

In some embodiments, the composition comprises no more than one bacterial component and the bacterial component of the composition consists of 1-20 bacterial species. Preferably, the bacterial component consists of 1, 2, 3 or 4 bacterial species. In other words, the composition does not contain any other bacterial species or comprises only de minimis or biologically irrelevant amounts of other bacterial species other than the bacterial species present in the bacterial component. In some embodiments, the bacterial component consists of bacteria:
(a) belonging to the species Enterococcus faecium;
(b) belonging to the subspecies Bifidobacterium longum subsp. infantis;
(c) belonging to the species Bifidobacterium breve; and/or
(d) belonging to the species Lacticaseibacillus rhamnosus.

In some embodiments, the bacterium belonging to the species Enterococcus faecium is the strain deposited under accession number DSM 22502 or a closely related strain thereof. In some embodiments, the bacterium belonging to the subspecies Bifidobacterium longum subsp. infantis is the strain deposited under accession number DSM 33867 or a closely related strain thereof. In some embodiments, the bacterium belonging to the species Bifidobacterium breve is the strain deposited under accession number DSM 33871 or a closely related strain thereof. In some embodiments, the bacterium belonging to the species Lacticaseibacillus rhamnosus is the strain deposited under accession number DSM 33870 or a closely related strain thereof. Any one or more of the bacterial strains disclosed herein may be the sole bacterial component of the composition (not taking into account de minimis or biologically irrelevant amounts of other bacterial strains or species).

The expression “closely related strain” as used above refers to a strain of the same species or subspecies that has similar phenotypic properties and a high degree of sequence identity (e.g., at least 99.0, 99.1, 99.2, 99.3, 99.4, 99.5, 99.6, 99.7, 99.7, 99.8 or 99.9% 16S rDNA sequence identity). The closely related strain may be the progeny of the reference strain. Such progeny may be the result of induced or random mutagenesis. The closely related strain maintains the therapeutic efficacy of the reference strain.

A combination of bacteria may be determined based on beneficial and compatible attributes of the probiotic strains in regards to; barrier function, exclusion of ETEC F18 to intestinal epithelial cells, growth in porcine milk oligosaccharides and inhibitory effects towards ETEC F18.

In some embodiments, the composition comprises at least 10⁴ CFU/g (colony forming units per gram) of each strain. In some embodiments, the composition comprises 10⁴ to 10¹¹ CFU/g of each strain. Preferably, the composition comprises 10⁶ to 10¹¹ CFU/g of each strain.

In some embodiments, the composition comprises at least 10⁴ CFU/g. In some embodiments, the composition comprises 10⁴ to 10¹¹ CFU/g. Preferably, the composition comprises 10⁶ to 10¹¹ CFU/g.

The composition of probiotic bacteria of the present disclosure may additionally comprise cryoprotectants, lyoprotectants, antioxidants, nutrients, fillers, flavorants or mixtures thereof. The composition may be in frozen or freeze-dried form. The composition preferably comprises one or more of cryoprotectants, lyoprotectants, antioxidants and/or nutrients, more preferably cryoprotectants, lyoprotectants and/or antioxidants and most preferably cryoprotectants or lyoprotectants, or both. Use of protectants such as croprotectants and lyoprotectantare known to a skilled person in the art. Suitable cryoprotectants or lyoprotectants include mono-, di-, tri- and polysaccharides (such as glucose, mannose, xylose, lactose, sucrose, trehalose, raffinose, maltodextrin, starch and gum arabic (acacia) and the like), polyols (such as erythritol, glycerol, inositol, mannitol, sorbitol, threitol, xylitol and the like), amino acids (such as proline, glutamic acid), complex substances (such as skim milk, peptones, gelatin, yeast extract) and inorganic compounds (such as sodium tripolyphosphate). Suitable antioxidants include ascorbic acid, citric acid and salts thereof, gallates, cysteine, sorbitol, mannitol, maltose. Suitable nutrients include sugars, amino acids, fatty acids, minerals, trace elements, vitamins (such as vitamin B-family, vitamin C). The composition may optionally comprise further substances including fillers (such as lactose, maltodextrin) and/or flavorants.

The term “cryoprotectant” as used herein, includes agents which provide stability to the strain against freezing-induced stresses, by being preferentially excluded from the strain's surface. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used.

In one embodiment, a lyoprotectant is added to a composition described herein. The term “lyoprotectant” as used herein, includes agents that provide stability to the strain during the freeze-drying or dehydration process (primary and secondary freeze-drying cycles), by providing an amorphous glassy matrix and by binding with the strain's surface through hydrogen bonding, replacing the water molecules that are removed during the drying process. This helps to minimize product degradation during the lyophilization cycle, and improve the long-term product stability. Non-limiting examples of lyoprotectants include sugars, such as sucrose or trehalose; an amino acid, such as monosodium glutamate, non-crystalline glycine or histidine; a methylamine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a composition is generally an amount that does not lead to an unacceptable amount of degradation of the strain when the composition is lyophilized.

In some embodiments, a bulking agent is included in the composition. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the strain stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in formulations in an amount from 0.5% to 10%.

Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the composition. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

The following items are preferred embodiments of the composition of the present invention (which may be combined with any embodiment discussed earlier):
1. A composition comprising probiotic bacteria.
2. The composition of item 1, wherein the composition comprises a bacterium which belongs to a genus selected from Enterococcus, Bifidobacterium, and Lactobacillus.
3. The composition of item 1 or 2, wherein the composition comprises a bacterium which belongs to the species Enterococcus faecium.
4. The composition of any one of items 1-3, wherein the composition comprises a bacterium which belongs to the species Bifidobacterium longum, preferably the subspecies Bifidobacterium longum subsp. infantis.
5. The composition of any one of items 1-4, wherein the composition comprises a bacterium which belongs to the species Bifidobacterium breve.
6. The composition of any one of items 1-5, wherein the composition comprises a bacterium which belongs to the species Lacticaseibacillus rhamnosus.
7. The composition of any one of items 1-6, wherein the composition comprises no more than 1 to 20 bacterial species.
8. The composition of any one of items 1-7, wherein the composition comprises at least 2, 3 or 4 bacterial species.
9. The composition of any one of items 1-8, wherein the composition comprises no more than 2 to 10, or 2 to 5 bacterial species.
10. The composition of any one of items 1-9, wherein the composition comprises no more than one bacterial component and the bacterial component of the composition consists of 1, 2, 3 or 4 bacterial species.

### Therapeutic Indication

In a further aspect, the present invention provides the composition of the invention (in accordance with any aspect/embodiment/disclosure described above) for use in a method of increasing resilience against infection by a pathogenic bacterium in a mammalian subject, wherein the method comprises administering the composition to the subject in the pre-weaning period.

In some embodiments, the composition is administered shortly after birth, e.g., within 12-16 hours after birth. In some embodiments, the composition is solely administered to the subject in the pre-weaning period (i.e., the composition is not administered during weaning or after weaning). The composition may be administered once or more than once.

In some embodiments, the subject is administered a dose of at least 10⁴, 10⁵, 10⁶, 10⁷, 10⁸, or 10⁹ CFU of each strain per day. Preferably, the subject is administered 10⁹ CFU or more of each strain per day. In some embodiments, the subject is administered a dose of at least 10⁴, 10⁵, 10⁶, 10⁷, 10⁸, or 10⁹ CFU of the probiotic bacteria per day. Preferably, the subject is administered 10⁹ CFU or more of the probiotic bacteria per day.

In some embodiments, the composition is administered orally or rectally. Preferably, the composition is administered orally. Oral administration can be achieved by using a drench gun.

In some embodiments, the method increases resilience against infection by a pathogenic bacterium in a post-weaning mammalian subject.

In some embodiments, the present invention provides a composition for use in a method of increasing resilience against infection by ETEC in a piglet, wherein the composition comprises no more than one bacterial component and the bacterial component consists of 1 to 10 or 2 to 5 bacterial species, and one of the bacterial species in the bacterial component is:
(i) Enterococcus faecium;
(ii) Bifidobacterium longum;
(iii) Bifidobacterium breve; and/or
(iv) Lacticaseibacillus rhamnosus.

In some embodiments, the present invention provides a composition for use in a method of increasing resilience against infection by ETEC in a piglet, wherein the composition comprises no more than one bacterial component and the bacterial component consists of 1 to 10 or 2 to 5 bacterial species, and one of the bacterial species in the bacterial component is Enterococcus faecium.

In some embodiments, the present invention provides a composition for use in a method of increasing resilience against infection by ETEC in a piglet, wherein the composition comprises no more than one bacterial component and the bacterial component consists of 1 to 10 or 2 to 5 bacterial species, and one of the bacterial species in the bacterial component is Bifidobacterium longum.

In some embodiments, the present invention provides a composition for use in a method of increasing resilience against infection by ETEC in a piglet, wherein the composition comprises no more than one bacterial component and the bacterial component consists of 1 to 10 or 2 to 5 bacterial species, and one of the bacterial species in the bacterial component is Bifidobacterium breve.

In some embodiments, the present invention provides a composition for use in a method of increasing resilience against infection by ETEC in a piglet, wherein the composition comprises no more than one bacterial component and the bacterial component consists of 1 to 10 or 2 to 5 bacterial species, and one of the bacterial species in the bacterial component is Lacticaseibacillus rhamnosus.

### Deposits and Expert Solution

The applicant requests that a sample of the deposited microorganisms stated below may only be made available to an expert, subject to available provisions governed by Industrial Property Offices of States Party to the Budapest Treaty, until the date on which the patent is granted or, where applicable, for twenty years from the date of filing if the application has been refused, withdrawn or is deemed to be withdrawn.

The applicant deposited the Enterococcus faecium strain on Apr. 22, 2009 at Leibniz Institute DSMZ—German Collection of Mikroorganisms and Cell Cultures, Inhoffenstrasse 7B, D-38124 Braunschweig and received the accession No. DSM 22502.

The applicant deposited the Bifidobacterium longum subsp. infantis strain on May 26, 2021 at Leibniz Institute DSMZ—German Collection of Mikroorganisms and Cell Cultures, Inhoffenstrasse 7B, D-38124 Braunschweig and received the accession No. DSM 33867.

The applicant deposited the Bifidobacterium breve strain on May 26, 2021 at Leibniz Institute DSMZ—German Collection of Mikroorganisms and Cell Cultures, Inhoffenstrasse 7B, D-38124 Braunschweig and received the accession No. DSM 33871.

The applicant deposited the Lacticaseibacillus rhamnosus strain on May 26, 2021 at Leibniz Institute DSMZ—German Collection of Mikroorganisms and Cell Cultures, Inhoffenstrasse 7B, D-38124 Braunschweig and received the accession No. DSM 33870.

The deposits have been made under the conditions of the Budapest Treaty on the International Recognition of the Deposit of Microorganisms for the Purposes of Patent Procedure.

### Examples

### Administration of Probiotic Compositions

### Animals and Housing

The study was conducted in three rounds with a total of 24 sows (Yorkshire×Landrace) mated with a Duroc boar. The sows were of 1-4 parity and tested homozygote carriers of the dominant gene coding for intestinal Enterotoxigenic Escherichia coli (ETEC) F18 fimbriae receptors. Sows were transported from the commercial farm to the research facility on day 85 of gestation, after which they were moved to the farrowing room on day 102 of gestation. The sows and piglets were housed in one farrowing room with eight loose farrowing pens (3.0×2.2 m) in two rows of four pens. The pens had partly slatted floor and were equipped with a covered creep area, an eating and drinking trough for the sow and a nipple drinker for the piglets. Furthermore, the pens were designed with farrowing rails and a sloped wall. Physical contact between pens was prevented by installing solid pen walls. The ventilation system was combi-diffuse, and the temperature was maintained at 20° C. during the first week, after which it was adjusted by 1° C. every week until a final temperature of 16° C. was reached. A heating lamp placed in the covered creep area was turned on before farrowing and kept on until seven days post farrowing. Additionally, extra heat for the piglets was provided the first seven days through floor heating in the covered creep area.

Easystrø (Easy-AgriCare A/S, Denmark), which is heat-treated chopped straw, was used as bedding in the covered creep area the first seven days after birth. Before farrowing, the sows had straw as bedding. After farrowing, the bedding was removed, but straw was allocated daily in a straw rack, and a rope was placed in each pen as investigation and manipulation activity for the sows. Sows were fed a standard sow pelleted diet with ingredient composition as described in Table 1. Sows were fed twice a day and daily rations were according to parity, stage of cycle and productivity. Piglets had no access to creep feed during suckling. All piglets were given iron supplementation.

TABLE 1
Ingredient composition of the sow diet.

| Ingredients | % |
|---|---|
| Barley | 41.13 |
| Wheat | 25.00 |
| Soybean meal | 13.30 |
| Oats | 5.00 |
| Wheat bran | 5.00 |
| Sunflower meal | 2.00 |
| Soybean hulls | 2.00 |
| Dried beet pulp | 2.00 |
| Calcium carbonate, chalk | 0.90 |
| Palm fatty acid distillate | 0.90 |
| Monocalcium phosphate | 0.65 |
| Vitamin and micromineral premixture | 0.55 |
| Sodium chloride | 0.53 |
| L-Lysine sulphate | 0.43 |
| Axtra XB (enzyme combination) | 0.20 |
| E vitamin mixed in wheat middling | 0.18 |
| Threonine 98% | 0.11 |
| DL-Methionine | 0.06 |
| Phytase enzyme (E4a24) | 0.06 |

### Preparation of Probiotic Inoculant

The composition consisted of four strains: Bifidobacterium (B.) longum subsp. infantis (DSM 33867), Bifidobacterium breve (DSM 33871), Lacticaseibacillus (L.) rhamnosus (DSM 33870) and Enterococcus (E.) faecium (DSM 22502). The composition included the four different probiotic strains in a 1:1 ratio (1×10⁹ CFU/strain/pig/day) blended with maltodextrin (0.35 g/pig/day). The maltodextrin and freeze-dried probiotic mixture were blended beforehand and divided into portions stored in airtight bags, one bag per litter per day. The placebo inoculant for the Control group only included the maltodextrin (0.35 g/pig/day). Placebo and probiotic mixtures were prepared right before each inoculation, by dissolving them in anaerobic phosphate buffered saline (pH 7.4) (2 mL/pig/day).

### Experimental Design

At farrowing, 24 litters were randomly allocated to two treatments and 168 piglets were included in each treatment group. Newborn piglets were orally inoculated with either placebo (Control group) or probiotics (Probiotic group). Inoculation was carried out maximum 16 hours after birth once all piglets in the respective litter had been born. Placebo or probiotics were administered to piglets once a day at 9 am the first four days after birth, and subsequently every second day until weaning on day 28. Inoculation was done using a Prima vaccinator device (Salfarm Denmark A/S) with a rubber tube. The first four days, the rubber tube was dipped in apple juice before inoculation. Each piglet in the Probiotic group was administered 4×10⁹ CFU dissolved in 2 mL anaerobic phosphate buffered saline and maltodextrin, whereas piglets in the Control group were administered with the same volume of anaerobic phosphate buffered saline and maltodextrin. Forty-eight hours after birth, litters were standardized to 16 piglets and five days after farrowing, litters were standardized to 14 piglets. In the standardization process, piglets excluded from the study were weak or previously treated with antibiotics. Cross-fostering was carried out, if necessary, within the first five days and only within treatment groups. All piglets were weaned at day 28 f 2 of age, and no probiotics were administered post-weaning until the end of the experiments at day 50 of age. On the day of weaning, three and two post-weaned piglets per litter from the Control and Probiotic group, respectively, were used for Example 1, and thus 8-9 post-weaned piglets per litter were used for Example 2.

### Example 1: Challenge with ETEC F18

### Treatments and Experimental Infection

A total of 60 weaned piglets (28+/−2 days of age) with an initial body weight of 9.1 f 1.7 kg were included in the experiment. The experiment was conducted over a 22-day period starting on the day of weaning. Pigs were allocated to three treatment groups; (i) Negative Control (non-challenged) (n=12), (ii) Positive Control (challenged) (n=24) and (iii) Probiotic (challenged and inoculated with probiotics during suckling) (n=24). On day 1 and 2 post-weaning, pigs in the Positive Control group and Probiotic group were challenged with ETEC F18 whereas pigs in the Negative Control group were provided with NaCl.

The challenge strain O138 F18-ETEC 9910297-2STM (positive for STb, LT, East-1, Stx2e, and F18ab) was isolated at the Danish Veterinary Institute (Frederiksberg, Copenhagen) from intestinal content of a pig with PWD. When grown on blood agar, the ETEC F18 was found to be hemolytic. The strain was grown aerobically in veal infusion broth at 37° C. for five hours with shaking (150 rpm) and OD₆₀₀ₙₘ 0.2 normalized in 0.9% NaCl. Each pig was challenged orally with 5 mL of viable ETEC F18 (5×10⁸ CFU/pig/day) on day 1 and 2 post-weaning. Pigs in the Negative Control group were provided with 5 mL of 0.9% NaCl.

### Feeding, Housing and Handling

During the experiment, piglets were fed a standard Danish nursery diet (Table 2) with ad libitum access through one feeder, and fresh water was permanently available through two drinking nipples. No straw was provided, but pigs had permanent access to ropes.

Two pigs from the same litter were housed together in 2.14 m×0.9 m pens with partially slatted floor and the concrete part of the floor had a cover and floor heating. The controlled environment unit was neutral pressure ventilated and linked to temperature sensors. At study start, the temperature was 24° C. and it was adjusted weekly until a final temperature of 19° C. For each of the three runs, pigs included in the experiment were housed in the same room, which had 16 pens.

TABLE 2
Ingredient composition of the nursery diet.

| Ingredients | % |
|---|---|
| Wheat | 53.21 |
| Barley | 21.00 |
| Soybean meal | 8.00 |
| ViloSoy, soy protein | 6.75 |
| Potato protein, protastar | 3.50 |
| Palm fatty acid distillate | 1.98 |
| Calcium carbonate, chalk | 1.25 |
| Monocalcium phosphate | 1.06 |
| Fish meal | 1.00 |
| Lysine sulphate 70 | 0.67 |
| Sugar beet molasses | 0.50 |
| Vitamin premixture | 0.40 |
| Feed salt, sodium chloride | 0.36 |
| Threonine 98% | 0.12 |
| DL-Methionine 98 | 0.11 |
| Tryptophane 99 | 0.04 |
| Ronozyme HiPhos | 0.03 |
| Valine L 96.5 | 0.02 |

The pigs challenged with ETEC F18 were housed in pens located next to each other and separated by three empty pens from the non-challenged pigs. Positive Control pigs were housed on the left side of the corridor and probiotics on the right side. To prevent bacterial cross-contamination, non-challenged pigs were always handled before ETEC-challenged pigs. Additionally, pigs in the Positive Control group were always handled before pigs in the Probiotic treatment group. When handling ETEC-challenged pigs, an extra layer of overalls and a special set of boots were worn. When moving from one pen to another, disposable gloves, aprons and plastic sleeves were changed. When weighing the pigs, plastic boxes assigned to each pen were used. Any physical contact between pigs from different pens was avoided by installing solid plastic walls between each pen.

### Registrations and Sample Collection

Rectal swaps were taken on weaning day (day 0), and thereafter daily during the first week of the experiment and three times a week during the last two weeks of the experiment. Samples were taken using a rubber glove lubricated in gel. The individual rectal swap samples were scored according to consistency following a 7 score scale (1: Hard, dry and lumpy; 2: Firm; 3: Soft but formable; 4: Soft and liquid; 5: Watery, dark; 6: Watery, yellow; 7: Yellow, foaming), where score 4-7 was considered as diarrhea. The rectal swap samples were stored on ice until being divided into aliquots for quantitative real-time Polymerase Chain Reaction (qPCR) (stored at −80° C.), and microbiological enumeration (conducted immediately).

### Microbiological Enumeration

For microbiological enumeration, 1-3 grams of fresh fecal matter was suspended in a peptone solution (1:10) and homogenized using a smasher paddle blender (bioMerieux Industry, USA) for two minutes. Serial dilutions were prepared and aliquots of 100 μL were added to agar plates for enumeration of Enterobacteriaceae on MacConkey (Merck 1.05465) and hemolytic colonies on blood agar plates (Oxoid Pb5039A). The plates were incubated overnight aerobically at 37° C., and CFU were counted using a manual colony counter. Blood agar plates with hemolytic colonies were stored at 5° C. until ETEC F18 serotyping was performed on five colonies per sample by the slide agglutination test using type-specific antisera (SSI Diagnostica A/S, Copenhagen, Denmark).

### Quantification of Est-II

Quantification of the gene encoding the heat-stable toxin STb (est-II) in fecal samples was carried out by qPCR. Standard curves for use in pig fecal samples were made by spiking known amounts of cells into pig feces. Standard curves were constructed from counted reference strain E. coli AUF18 (9910297-2STM) (serotype 0138:F18, virotype F18ab STb, LT, EAST1, and Stx2e) spiked into feces from a healthy adult pig that did not contain any ETEC F18. Well-defined single colonies of AUF18 grown on Luria-Bertani (LB) media were transferred from solid media to broth media. Cultures were grown overnight, and cells were pelleted by centrifugation, and subsequently the pellet was resuspended in 400 μL of PBS and then serially diluted in PBS. The cells were counted from an appropriate dilution in a Bürker-Turk counting chamber where the average of 5 squares (0.2 by 0.2 mm) were used to calculate the original cells per mL. Standard curves were made by spiking 100 μL of 50% feces (diluted 50% with PBS buffer) with 100 μL of cell suspensions of the different reference bacteria in 5-fold dilutions prior to DNA extraction. Feces samples stored at −80° C. were defrosted and weighed for DNA extraction. DNA was extracted using E.Z.N.A Stool DNA kit (Omega bio-tek, Norcross, GA, USA) using the manufacturers method for ‘DNA extraction and purification from stool for pathogen detection’ with the following modifications. Samples were disrupted after addition of SLX-buffer in a star-beater (VWR) at frequency 30 (1/s) for five minutes. DNA was eluted in 200 μL of elution buffer for the spiked standards and 100 μL of elution buffer for the samples and stored at −20° C. until further analysis. DNA concentration was determined using a Qubit Fluorometer.

Quantitative real-time PCR was performed on a ABI ViiA7 real-time PCR system (Thermo Fisher Scientific) using MicroAmp Optical 384 well reaction plate (Applied Biosystems). Quantitative real-time PCR reactions contained 5 μL of Maxima SYBR Green/ROX qPCR Master Mix (Thermo Scientific), STb primers at a concentration of 0.3 mM, 2 μL of template DNA and water to a final volume of 10 μL.

The primer combination for the STb gene was forward 5′-TGCCTATGCATCTACACAAT-3′ (SEQ ID NO: 5) and reverse 5′-CTCCAGCAGTACCATCTCTA-3′ (SEQ ID NO: 6).

All assays contained a standard curve and a no template control and were performed in triplicate. Conditions of the PCR were as follows: pre-treatment 2 min at 50° C., initial denaturation 10 min at 95° C., 40 cycles of denaturation 30 s at 95° C. For the STb gene, annealing and extension were 60 s at 59.1° C. Melting curves were generated by increasing the temperature from 60° C. to 95° C. at a rate of 0.05° C./s recording continuously. Target concentrations were calculated using the QuantStudio realtime PRC software that comes with the machine from Ct values. The detection limit was Ct values greater than 32 corresponding to 10⁵ cells/g feces.

### Results

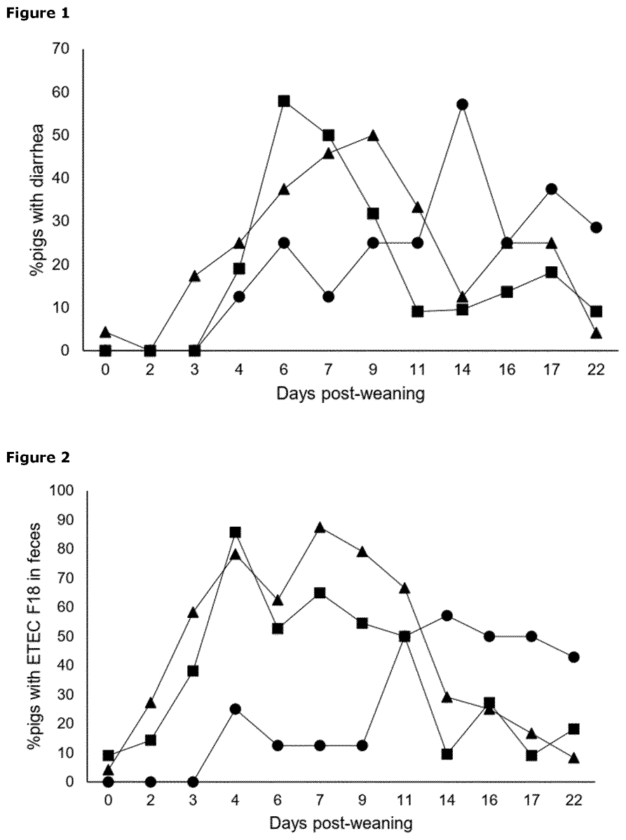

The course of event when looking at diarrhea incidences and presence of ETEC F18 in feces pointed to the Probiotic group having a more rapid response towards the pathogen challenge and coping with the pathogen challenge faster compared with the Positive control group (FIG. 1). Thus, the treated piglets suffered for fewer days with diarrhea.

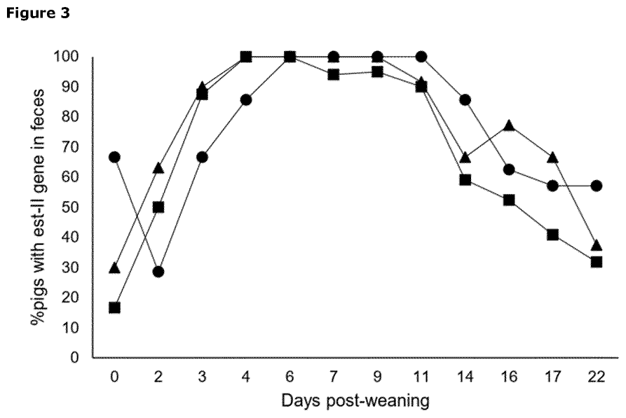

Odds ratio results showed that the Positive Control group had 83% higher risk of having ETEC F18 present in feces compared with the Probiotic group (p=0.004) during the entire study (FIG. 2). There were fewer pigs in the Probiotic group shedding ETEC F18 on day 9 (p=0.02) than in the Positive Control group; and the same tendency was observed on day 7 (p=0.08) and 14 (p=0.07). Looking the est-II gene (heat-stabile toxin Stb), the Probiotic group had significantly lower number of pigs with detectable concentration of the est-IH gene in feces compared with the Positive Control group (p=0.04, see FIG. 3).

The findings of this study demonstrate that administration of probiotics early in life resulted in beneficial effects when piglets were subjected to an ETEC F18 challenge post-weaning compared with ETEC F18 challenged pigs not inoculated with probiotics during suckling. In comparison with the Positive Control group, the beneficial effect of early probiotic inoculation on ETEC F18 challenged pigs was expressed by a reduction of fecal shedding of F18 and its toxin as well as in a decrease in the number of days that the treated group suffered from diarrhea.

### Example 2: Non-Challenged Set-Up

### Animals and Housing

After weaning, litter mates were housed together in the same nursery pen. Seven days after weaning and after selecting two pigs per pen for slaughter, pens were adjusted to maximum five pigs by euthanizing pigs which were either weak or previously treated with antibiotics. The nursery room contained eight pens (2.1×1.8 m) in two rows of four. Pens had partially slatted floor and the concrete part of the floor had a covering and floor heating. The unit was neutral pressure ventilated linked to temperature sensors. At study start, the temperature was 24° C. and it was adjusted by ˜1.5° C. every week until a final temperature of 19° C. was reached. Nursery piglets were fed a nursery pelleted feed through two feeders with ad libitum access. The feed was a standard Danish nursery diet with ingredient composition as described in Table 2. Fresh water was accessible through four drinking nipples. No straw was provided, but pigs had permanent access to ropes as an investigation and manipulation activity. Physical contact between pigs from different pens was prevented by installation of solid pen walls. To prevent bacterial cross contamination between treatment groups, pigs in the Control group were always handled before pigs in the probiotic group. When entering a pen or handling pigs, disposable gloves, shoe covers, aprons, and plastic sleeves were used. Plastic boxes assigned to each pen were used when weighing the pigs.

### Registrations and Sample Collection

If piglets were treated with antibiotics, the reason was noted down, and these piglets were not included in collection of samples subsequently. Occurrence of diarrhea in each pen was assessed daily during the entire experiment according to the method of Toft and Pedersen, 2011. Prev Vet Med. 98(4):288-91. Scores were 1: Firm and shaped; 2: Soft and shaped; 3: Loose; 4: Watery. Diarrhea was defined as score 3 or 4, and diarrhea incidence (%) was calculated as number of pens with diarrhea (score 3 or 4) out of total number of days.

Three days after birth, three median piglets per litter were selected for collection of feces. These piglets were followed during the entire experiment by taking a rectal swap on day 3, 7, 14, 21, 28, 35, 42 and 50. Rectal swaps were collected using a cotton bud dipped in gel and samples were kept on ice until storage. Samples were stored at −20° C. or −80° C. dependent on further analysis.

On day 23-24 and day 35-36, two median pigs per litter were selected for blood sampling and for slaughter. Blood samples from the jugular vein were collected in EDTA containing—vacutainers for hematology analysis. Blood was analyzed immediately after collection. After blood sampling, pigs were euthanized using a captive bolt gun followed by bleeding. The gastrointestinal tract was removed, digesta content weighed and pH measured. The small intestine was divided into two (proximal and distal), and colon was divided into three segments of equal length (proximal, mid, and distal). Digesta (stomach, proximal and distal small intestine, cecum, proximal, mid, and distal colon) from each of the two pigs per litter was pooled by taking the same amount from each pig and stored at −80° C. until further analysis. Mucosal samples were taken from proximal and distal small intestine and proximal colon. Before sampling, the intestines were rinsed with sterile phosphate buffered saline several times to remove digesta and free-floating bacteria. Then samples were collected by gently scraping off the mucosa from the epithelial layer by using a sterile glass microscope slide. Samples were kept in fluid nitrogen until being stored at −80° C. until further analysis.

### Analytical Methods

For the gene expression analysis, total RNA was extracted from the distal small intestinal mucosal scrapings of the individual pigs (not pooled from two pigs) using the NucleoSpin RNA kit (Ref. 740955 Macherey-Nagel, Germany) including DNAse treatment. RNA was extracted following the instructions of the manufacturer with a pre-step homogenizing the samples for 2×2 min with a steel ball. Complementary DNA (cDNA) was synthesized from 1000 ng RNA using the High-Capacity cDNA Reverse Transcription Kit (Ref. 4368813, Applied Biosystems, USA) according to the manufacturer's instructions. High-throughput quantitative real-time PCR was performed using the 192.24 dynamic array integrated fluidic circuits (Fluidigm, South San Fransisco, Calif) following methods previously described by Skovgaard et al., 2013. Innate Immun. 19(5):531-44 with minor modifications including 18 cycles of pre-amplification. qPCR was performed by combining 82 pre-amplified samples with 22 primer sets. Primer sequences and amplicon length for each assayed mRNA gene are listed in Additional file 1. Data were corrected for PCR efficiency for each primer assay individually and subsequently normalized using the average reference gene expression of three reference genes: Glyceraldehyde-3-Phosphate Dehydrogenase (GAPDH), Peptidylprolyl isomerase A (PPIA), and TATA-Box Binding Protein (TBP). The three reference genes were confirmed to be suitable endogenous reference genes, as they were not affected by the treatment. Normalized C_{q} values for each of the genes were converted to relative quantities by calculating 2^{(highest_assay_Cq-actual_sample_Cq)}, so that the sample with the highest C_{q} (lowest gene expression) was given a value of 1 and all other samples values >1 as described by Brogaard et al. 2015. BMC Genomics. 16(1):417.

For 16S rRNA amplicon sequencing, feces, mucosa and digesta samples stored at −80° C. were thawed and weighed. DNA was extracted from 100 mg of feces and digesta samples using the E.Z.N.A.® Stool DNA Kit (Omega Bio-tek, USA) following the instructions of the manufacturer with the exception of including a steel ball for homogenization until the step, where the supernatant is aspirated. For mucosa samples, 12.5 mg from each of the two pigs per litter were pooled to extract DNA using the NucleoSpin Tissue DNA kit (Macherey-Nagel, Germany) according to manufacturer instructions. DNA was diluted to 1 ng/μL using sterile water. The V3-V4 region of the 16S rRNA genes was amplified using the 341F and 806R primer disclosed in, for example, Behrendt et al., 2012. ISME J. 6(6): 1222-1237. All PCRs were conducted using the Phusion® High-Fidelity PCR Master Mix (New England Biolabs). Agarose gel (2%) electrophoresis was performed to verify amplicon size using a 1× loading buffer (contained SYB green). Samples with bright main bands between 400 bp-450 bp were chosen for further analyses. PCR products were mixed at equal density ratios and purified with Qiagen Gel Extraction Kit (Qiagen, Germany). Libraries were generated with the NEBNext® Ultra™ DNA Library Prep Kit for Illumina (New England Biolabs, Inc, USA), quantified via Qubit and qPCR and submitted to sequencing. Sequencing was performed on an Illumina NovaSeq 6000 platform generating 2×250 bp paired-end sequence reads. Library preparation and sequencing was carried out by Novogene, UK.

### Microbiome Data Analysis

Illumina MiSeq fastq files were processed using USEARCH (v.11.0). Raw reads were merged, trimmed, and quality filtered using the fstq_mergepairs and the fastq_filter scripts implemented in the USEARCH pipeline as previously described by Krych et al., 2018. J Micrbiol Methods. 144:1-7. The UNOISE3 algorithm with default settings was applied to denoise data, purge chimeric reads, and construct zero-radius operational taxonomic units (zOTU). Taxonomic assignment of zOTUs was performed with SINTAX (Edgar, 2016. bioRxiv. 081257) using the Greengenes (13.8) 16S rRNA gene collection reference database. Subsequent analysis steps were carried out using R (version 3.6.0). zOTUs unassigned at phylum or class level, zOTUs assigned as chloroplasts, mitochondria, cyanobacteria, elusimicrobia, planctomycetes or verrucomicrobia, as well as zOTUs present in less than two samples and with a total abundance less than 0.001% across all samples were removed. Uneven sampling depth was normalized by rarefication to a read depth of 15000 reads per sample using the Phyloseq package (version 1.30.0) (McMurdie & Holmes, 2013. PLoS One. 8(4):e61217), discarding 57 out of 665 samples (see rarefaction curve in Additional file 2). If not stated differently, subsequent microbiome analyses were conducted for filtered and rarefied data subdivided based on sample type (feces, mucosa, digesta), sampling day (feces: d3, d7, d14, d21, d28, d35, d42, d50; mucosa and digesta: d23-24, d35-36) and sampling location within the gastrointestinal tract (mucosa and digesta: small intestine, proximal colon), separately. Microbial diversity analyses were performed using the packages Phyloseq and Vegan (available at: https://cran.r-project.org/package=vegan). For alpha diversity, observed number of zOTUs and the Shannon's diversity index were calculated. Satisfaction of normality was tested using the Shapiro-Wilk test and effects of treatment and round on alpha diversity were investigated by linear mixed-effects models. Linear mixed-effects models were conducted using the Imer function implemented in the Ime4 package (Bates et al., 2015. J Stat Softw. 67(1):1-91), with treatment and round as fixed effects and sow as random effect. For beta diversity, Bray-Curtis dissimilarity distances were estimated. Based on Bray-Curtis distances a principle coordinate analysis (PCoA) was performed and PCoA ordination plots were generated using the ggplot2 package (version 3.3.1). To investigate the effect of treatment group on beta diversity, a permutational multivariate analysis of variance (PERMANOVA) on Bray-Curtis distances was performed using the adonis function implemented in the Vegan package. Since adonis is not able to account for confounding factors, a potential confounding effect of round was tested by PERMANOVA in each of the sub-datasets. If round was found to be significant, data were further divided based on round, otherwise data for the three rounds were analyzed combined. Homogeneity of group dispersions (variance) was verified using the betadisper function implemented in Vegan, and a nested PERMANOVA on Bray-Curtis distances with sow nested within treatment group was carried out for each sub-dataset separately, using the function nested.npmanova on Bray-Curtis distances based on the adonis algorithm implemented in the biodiversityR package (version 2.12-3) and applying 999 permutations. Differential abundance analysis was carried out to identify community differences between treatment groups using the DESeq2 package (version 1.2.6) with filtered but not rarefied data. zOTU counts were normalized using the variance-stabilizing transformation approach implemented in DESeq2 and pseudo-counts of one were added to zero zOTU counts as previously described by McMurdie and Holmes (see McMurdie & Holmes, 2013. PLoS One. 8(4):e61217). zOTUs were included in the results if the Log2 fold change >2 and if the adjusted P-value was 0.01. The ampvis2 package was used to generate heatmaps of the 15 most abundant families.

### Results

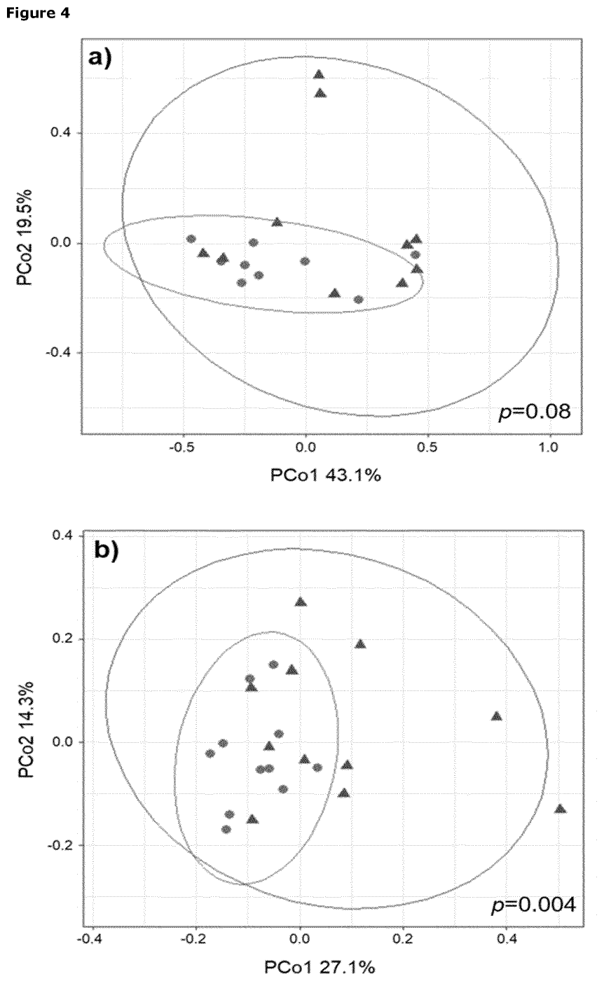

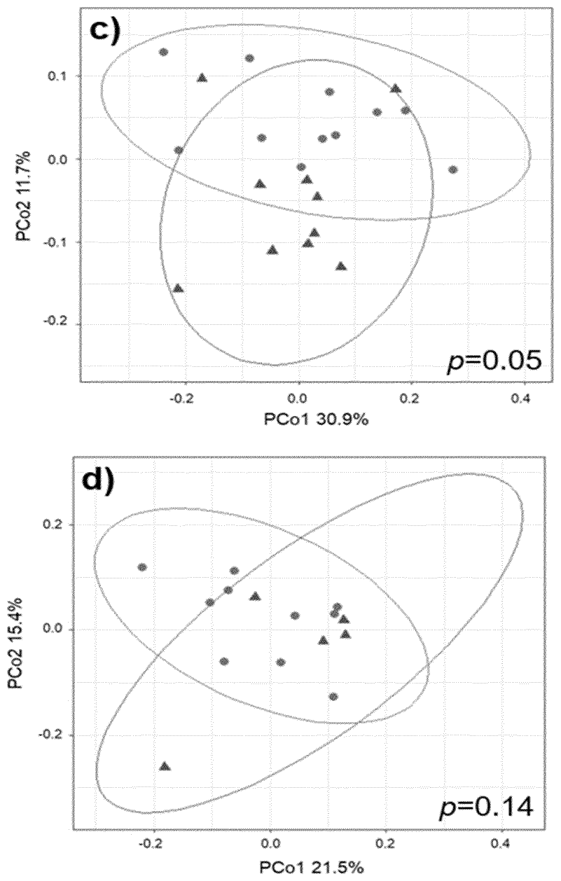

In a parallel study using the piglets administered with probiotics or placebo during suckling, the effect of early probiotic inoculation after cessation of its administration in weaned pigs under a commercial and non-challenged setup was assessed. Microbiota composition was analyzed in feces and in intestinal content (digesta and mucosa). Nested PERMANOVA on Bray-Curtis distance metrics analysis (see, for example, Anderson, 2017. Wiley StatsRef: Statistics Reference Online. doi: 10.1002/9781118445112.stat07841) demonstrated alterations in microbial diversity between the two treatment groups (placebo and probiotics) on day 35 after cessation of probiotic administration. A striking shift was observed between the two treatment groups on day 35 in digesta from the ascending colon, small intestinal mucosa and in feces (see FIG. 4).

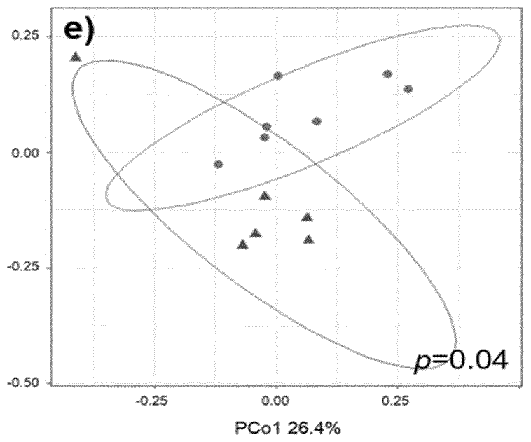

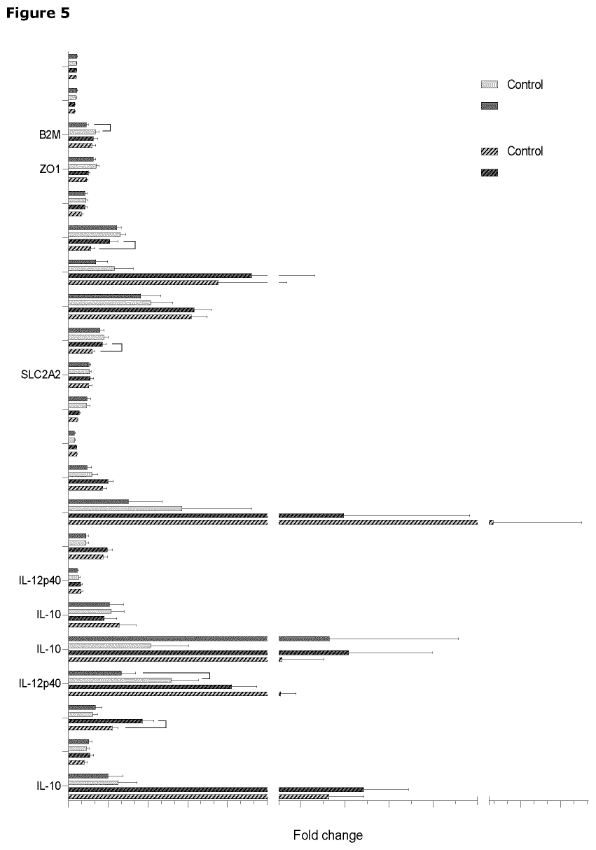

Gene expression in small intestinal mucosa pre- and post-weaning was analyzed (see FIG. 5). The probiotic pigs seemed to have a more local expression of genes post-weaning (i.e. MUC2, pro-inflammatory cytokines IL8, and IL17). The placebo group on the other hand prevailed a higher expression of the acute phase protein SAA, especially post-weaning, which may be an expression of insufficient local defenses inducing a systemic response. It is well established that SAA levels are significantly elevated after weaning, and that an elevation of acute phase proteins including SAA is an early systemic sign of disease. In combination with a high expression of SAA in small intestinal mucosa, the placebo group also had significantly more pens with diarrhea the first week post-weaning (see FIG. 6).

In summary, it may be deduced that piglets supplemented with probiotics early in life and during suckling may be better at overcoming the weaning process (even after cessation of probiotic administration), possibly through increased local mucosal immune response due to early probiotic priming. These mechanisms may explain part of the mechanisms making the early probiotic administered pigs more resilient towards ETEC infections.

### Example 3: E. faecium Counteracts the Transepithelial Electrical Resistance (TEER) Decrease Caused by ETEC F4

### Material and Methods

E. faecium was inoculated from frozen stock and incubated aerobically overnight at 37° C. in De Man, Rogosa and Sharpe (MRS) broth, pH 6.5 (Difco™, 288,110, Chr. Hansen A/S Denmark). Ten-fold dilution series were prepared from the overnight cultures and incubated under the same conditions as described above. Late exponential/early stationary phase, reached after 18 h of incubation, was selected for the assays based on measures of optical density at 600 nm (OD₆₀₀).

The ETEC strain Abbotstown serotype 0149:K91:F4ac was chosen as the challenge strain for the TEER assay. ETEC F4 is considered as one of two common fimbria types responsible for PWD in nursery pigs (Luise et al., 2019. J Anim Sci Biotechnol. 10:53), and the serotype has previously been associated with diarrhea in newly weaned pigs (Frydendahl, 2002. Vet Microbiol. 85(2):169-82; Nadeau et al., 2017. Vet J. 226:32-39). The ETEC F4 challenge strain was inoculated from frozen stock and incubated overnight in Luria-Bertani (LB) broth, pH 7.0.

Caco-2 cell monolayers were equilibrated overnight in antibiotic-free cell culture medium with hourly TEER measurements using a CellZscope2 system (NanoAnalytics, Germany). On the day of the experiment, E. faecium and ETEC F4 grown as described above to late-exponential phase (E. faecium) or stationary phase (ETEC F4) were washed and resuspended in antibiotic-free cell culture medium. OD₆₀₀-normalized bacteria were then added to the apical compartments of the cell monolayers at a concentration of approximately 10⁸ CFU/well and 10⁷ CFU/well for E. faecium and ETEC F4, respectively, after which hourly TEER measurements were carried out for 12 h. The assay was performed in triplicate and repeated twice.

### Results

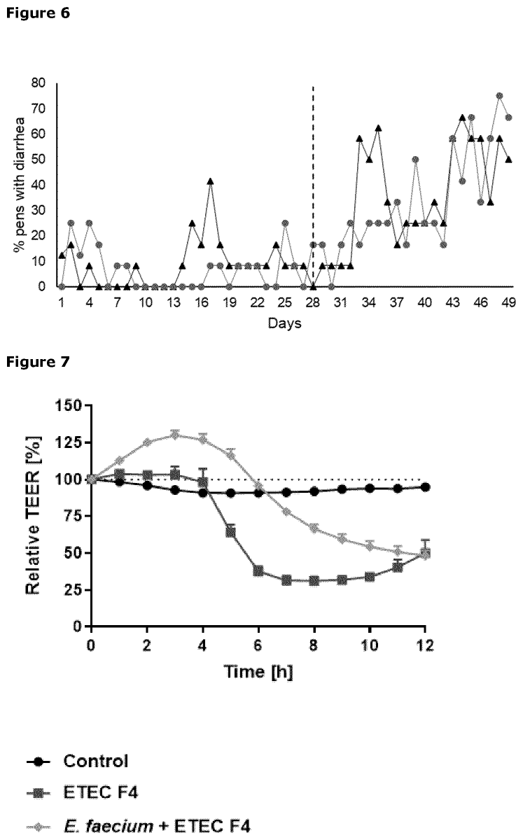

ETEC F4 quickly caused damage to the cells with TEER dropping to approx. 50% of the initial level after 5 h and 25% after 8 h (see FIG. 7). E. faecium counteracted the TEER decrease to high extent.

### Example 4: E. faecium Stimulates Dendritic Cells (DCs) to Release IL-10 and IL-12

### Materials and Methods

On day 0, the buffy coat(s) were picked up from the hospital's blood bank. The blood was transported at ambient temperature (no ice). For the experiment, the buffy coat (˜60 mL) was transferred to a sterile T75 cell culture flask and diluted to 120 mL with DC medium (RPMI supplemented with 50 μM 2-mercaptoethanol, 10 mM HEPES and penicillin-streptomycin, pre-warmed to 37° C.). 15 mL Ficoll-Paque was carefully distributed in each of four 50 mL SepMate tubes. 30 mL diluted buffy coat was carefully placed on top of the SepMate tubes and centrifuged at 1200×g, 10 min, 25° C., with brake. The upper layer was transferred to clean 50 mL tubes (4 in total) by pouring the liquid quickly and steady. DC medium was added to a final volume of 45 mL in each 50 mL tube and spun 700×g, 10 min, room temperature (RT). Afterwards the supernatant was discarded, and the pellet was resuspended in 5 mL DC medium, after which it was pooled into one 50 mL tube. If the buffy coat contained clumps, the cell suspension was passed through a 70 μm cell strainer. DC medium was added to a final volume of 45 mL in each 50 mL tube and spun 300×g, 10 min, RT. The supernatant was discarded and the pellet was resuspended in 2.5 mL PBSE solution (PBS supplemented with 2 mM EDTA, 0.5% fetal bovine serum, penicillin-streptomycin) (final volume). 40 μL of human CD14+ microbeads/10⁸ cells (maximum 200 μL) was added and the bead volume was adjusted to desired DC yield. It was incubated 30 min at 4° C. 22.5 mL PBSE solution was added and the suspension was spun at 300 g, 10 min, RT. The supernatant was discarded, and the pellet was resuspended in 3 mL PBSE solution. One LS MidiMACS column was placed in the magnet and a sterile 50 ml tube was used to collect flow through. The column was washed with 3 mL PBSE solution, after which the cell suspension was applied to the column. The column was then rinsed with 3×3 mL PBSE solution and afterwards it was removed from the magnet. The positive fraction was collected into a 50 mL tube by adding 5 mL PBSE solution to column and pressing out the CD14+ cells using the plunger. The DC medium (complete) (DC medium supplemented with 10% fetal bovine serum and 2 mM L-glutamine) was added to 30 mL total volume, and the cells were counted and spun at 300 g, 10 min, RT. The supernatant was discarded, and the pellet was resuspended at 2×10⁶ cells/mL in DC medium (complete) containing 30 ng/mL IL-4 and 20 ng/mL GM-CSF. IL-4 and GM-CSF stocks were both 100 μg/mL. Cells were plated at 3 mL/wells in 6-well plates and incubated at 37° C., 5% CO₂.

On day 3, 1 mL culture supernatant was removed from each well and 1.5 mL of freshly prepared DC medium (complete) supplemented with 30 ng/mL IL-4 and 20 ng/ml GM-CSF was added to each well. On day 6, cells were harvested by gentle collection, and DCs were counted. Then the solution was spun at 300 g, 10 min, RT, and the supernatant was discarded. The pellet was resuspended in antibiotics-free DC medium (complete) (No IL-4 or GM-CSF) at desired concentrations and seeded in 96-well plates.

The cell density was adjusted to 1.25×10⁶ cells/mL. 80 μL was distributed per well (for a final concentration of 1×10⁵ DCs/well) and incubated >1 hr at 37° C., 5% CO₂. 20 μL of DC medium (complete) (negative control) or bacterial cultures (for a final concentration of approx. 1×10⁶ CFU/well) were then added, after which the plate was incubated for 20 h at 37° C., 5% CO₂.

On day 7, the cell culture supernatants were collected from the wells. 70 μL supernatant was transferred to AcroPrep 96-well plate placed on top of a regular 96-well plate. The supernatants were filtered by centrifugation at 1,500 g, 10 min into the regular 96-well plate. Immediately after, the 96-well plate containing the filtered supernatants was frozen and stored at −80° C. until used for cytokine profiling. Levels of IL-10 and IL-12 was quantified using the U-PLEX platform (Meso Scale Discovery (MSD), US) according to manufacturer's instructions.

### Results

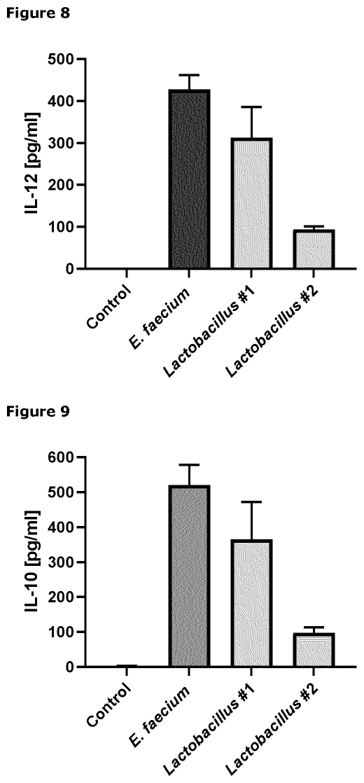

Interleukin-12 (IL-12) is produced by dendritic cells and other immune cells in response to antigenic stimulation. It is involved in the differentiation of naïve T cells into Th1 helper T cells. It also plays an important role in enhancing the cytotoxic activity of other types of immune cells specialized in killing infected cells (Heufler et al., 1996. Eur J Immunol. 26(3):659-68). The results showed that E. faecium stimulates the dendritic cells to release large amounts of IL-12 (see FIG. 8). This highlights the potential of E. faecium to shift a dominance of Th2 helper T cells towards a balanced state.

Interleukin-10 (IL-10) is produced primarily by monocytes, dendritic cells and macrophages It has multiple functions, but is overall an anti-inflammatory cytokine. It is involved in the differentiation of naïve T cells into regulatory T cells (Tregs), which in turn play a crucial role in maintaining tolerance to self-antigens and prevent autoimmune diseases (Alameddine et al., 2019. Front Immunol. 10:143). Results showed that E. faecium stimulates the dendritic cells to release large amounts of IL-10 (see FIG. 9). This highlights the potential of E. faecium to drive T cells to become regulatory T cells thus creating tolerance. In addition, the production of IL-10 by dendritic cells exposed to E. faecium highlights the potential anti-inflammatory properties of E. faecium in vivo.

E. faecium was superior in stimulating the dendritic cells to release IL-10 and IL-12 compared with the two Lactobacillus strains tested.

The results of Examples 3 and 4 suggest that a beneficial effect could be obtained by using a composition containing just E. faecium. Further, these results suggest that administration of a composition comprising a single bacterial strain, such as a E. faecium strain, during the pre-weaning period would have a positive therapeutic effect on the subject.

1. A method of increasing resilience against infection by a pathogenic bacterium in a mammalian subject, comprising administering a composition comprising probiotic bacteria to the subject in a pre-weaning period.

2. The method according to claim 1, wherein the composition is solely administered to the subject in the pre-weaning period.

3. The method according to claim 1, wherein the composition comprises a bacterium which belongs to a genus selected from Enterococcus, Bifidobacterium, and Lacticaseibacillus.

4. The method according to claim 1, wherein the composition comprises a bacterium of species Enterococcus faecium.

5. The method according to claim 1, wherein the composition comprises a bacterium of species Bifidobacterium longum.

6. The method according to claim 1, wherein the composition comprises a bacterium of species Bifidobacterium breve.

7. The method according to claim 1, wherein the composition comprises a bacterium of species Lacticaseibacillus rhamnosus.

8. The method according to claim 1, wherein the composition comprises no more than 20 bacterial species.

9. The method according to claim 1, wherein the composition comprises at least 2 bacterial species.

10. The method according to claim 1, wherein the composition comprises no more than 10 bacterial species.

11. The method according to claim 1, wherein the composition comprises no more than one bacterial component, wherein the bacterial component consists of 1, 2, 3 or 4 bacterial species.

12. The method according to claim 1, wherein the method increases resilience against infection by a pathogenic bacterium in a post-weaned mammalian subject.

13. The method according to claim 1, wherein the mammalian subject is a piglet.

14. The method according to claim 1, wherein the pathogenic bacterium is an E. coli strain.

15. The method according to claim 1 wherein:
the composition comprises no more than one bacterial component, wherein the bacterial component consists of 1 to 10 bacterial species, wherein one of the bacterial species in the bacterial component is Enterococcus faecium;
the pathogenic bacterium is an enterotoxigenic E. coli strain;
the method increases resilience against infection by a pathogenic bacterium in a post-weaned mammalian subject; and/or
the mammalian subject is a piglet.

16. The method according to claim 5, wherein the composition comprises a bacterium of subspecies Bifidobacterium longum subsp. infantis.


SEQUENCE LISTING (6 sequences)

SEQ ID NO: 1 - DNA, 1477 bp, Artificial Sequence
  16S rDNA sequence of E. faecium
taatacatgcaagtcgaacgcttctttttccaccggagcttgctccaccggaaaaagaggagtggcgaacgggtgagtaacacgtgggtaacctgcccatcagaaggggataacacttggaaacaggtgctaataccgtataacaatcgaaaccgcatggttttgatttgaaaggcgctttcgggtgtcgctgatggatggacccgcggtgcattagctagttggtgaggtaacggctcaccaaggccacgatgcatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttgttagagaagaacaaggatgagagtaactgttcatcccttgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagcttccccttcgggggcaaagtgacaggtggtgcatggttgtcgtcagctcgtgtcgtgagatgttgggttaagtcccgcaacgagcgcaacccttattgttagttgccatcattcagttgggcactctagcaagactgccggtgacaaaccggaggaaggtggggatgacgtcaaatcatcatgccccttatgacctgggctacacacgtgctacaatgggaagtacaacgagttgcgaagtcgcgaggctaagctaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaaccttttggagccagccgcctaaggtgggatagatgattggggtgaagtcgtaacaaggta

SEQ ID NO: 2 - DNA, 1565 bp, Artificial Sequence
  16S rDNA sequence of L. rhamnosus
agagtttgatcatggctcaggatgaacgctggcggcgtgcctaatacatgcaagtcgaacgagttctgattattgaaaggtgcttgcatcttgatttaattttgaacgagtggcggacgggtgagtaacacgtgggtaacctgcccttaagtgggggataacatttggaaacagatgctaataccgcataaatccaagaaccgcatggttcttggctgaaagatggcgtaagctatcgcttttggatggacccgcggcgtattagctagttggtgaggtaacggctcaccaaggcaatgatacgtagccgaactgagaggttgatcggccacattgggactgagacacggcccaaactcctacgggaggcagcagtagggaatcttccacaatggacgcaagtctgatggagcaacgccgcgtgagtgaagaaggctttcgggtcgtaaaactctgttgttggagaagaatggtcggcagagtaactgttgtcggcgtgacggtatccaaccagaaagccacggctaactacgtgccagcagccgcggtaatacgtaggtggcaagcgttatccggatttattgggcgtaaagcgagcgcaggcggttttttaagtctgatgtgaaagccctcggcttaaccgaggaagtgcatcggaagctggaaaacttgagtgcagaagaggacagtggaactccatgtgtagcggtgaaatgcgtagatatatggaagaacaccagtggcgaacgcggctgtctggtctgtaactgacgctgaggctcgaaagcatgggtagcgaacaggattagataccctggtagtccatgccgtaaacgatgaatgctaggtgttggagggtttccgcccttcagtgccgcagctaacgcattaagcattccgcctggggagtacgaccgcaaggttgaaactcaaaggaattgacgggggcccgcacaagcggtggagcatgtggtttaattcgaagcaacgcgaagaaccttaccaggtcttgacatcttttgatcacctgagagatcaggtttcccctccgggggcaaaatgacaggtggtgcatggttgtcgtcagctcgtgtcgtgagatgttgggttaagtcccgcaacgagcgcaacccttatgactagttgccagcatttagttgggcactctagtaagactgccggtgacaaaccggaggaaggtggggatgacgtcaaatcatcatgccccttatgacctgggctacacacgtgctacaatggatggtacaacgagttgcgagaccgcgaggtcaagctaatctcttaaagccattctcagttcggactgtaggctgcaactcgcctacacgaagtcggaatcgctagtaatcgcggatcagcacgccgcggtgaatacgttcccgggccttgtacacaccgcccgtcacaccatgagagtttgtaacacccgaagccggtggcgtaacccttttagggagcgagccgtctaaggtgggacaaatgattagggtgaagtcgtaacaaggtagccgtaggagaacctgcggctggatcacctcctttct

SEQ ID NO: 3 - DNA, 1485 bp, Artificial Sequence
  16S rDNA sequence of B. breve
ttcgattctggctcaggatgaacgctggcggcgtgcttaacacatgcaagtcgaacgggatccatcgggctttgcttggtggtgagagtggcgaacgggtgagtaatgcgtgaccgacctgccccatgcaccggaatagctcctggaaacgggtggtaatgccggatgctccatcacaccgcatggtgtgttgggaaagcctttgcggcatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgttagggagcaaggcactttgtgttgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacagtgcgagctggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgcgggagggagccgtctaaggtgaggctcgtgattgggactaagtcgtaacaaggta

SEQ ID NO: 4 - DNA, 1492 bp, Artificial Sequence
  16S rDNA sequence of Bifidobacterium longum subsp. infantis
ggctcaggatgaacgctggcggcgtgcttaacacatgcaagtcgaacgggatccatcgggctttgcttggtggtgagagtggcgaacgggtgagtaatgcgtgaccgacctgccccatacaccggaatagctcctggaaacgggtggtaatgccggatgttccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgtgagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacgatcccagagatggggtttcccttcggggcgggttcacaggtggtgcatggtcgtcgtcagctcgtgtcgtgagatgttgggttaagtcccgcaacgagcgcaaccctcgccccgtgttgccagcggattgtgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggct

SEQ ID NO: 5 - DNA, 20 bp, Artificial Sequence
  STb gene forward
tgcctatgcatctacacaat

SEQ ID NO: 6 - DNA, 20 bp, Artificial Sequence
  STb gene reverse
ctccagcagtaccatctcta